(19) United States
(12) Patent Application Publication
(10) Pub. No.: US 2024/0002789 A1
(43) Pub. Date: Jan. 4, 2024

(54) SKIN SPHEROIDS AND PROCESS OF PREPARATION AND USE THEREOF

(71) Applicants: The Governing Council of The University of Toronto, Toronto (CA); BASF SE, Ludwigshafen am Rhein (DE); Ted W. DEISENROTH, Tarrytown, NY (US)

(72) Inventors: Eugenia KUMACHEV, Toronto (CA); Valerie ANDRE, Lyon Cedex (FR); Ted W. DEISENROTH, Tarrytown, NY (US); Zhengkun CHEN, Toronto (CA)

(21) Appl. No.: 18/037,740

(22) PCT Filed: Nov. 5, 2021
(86) PCT No.: PCT/US2021/058313

§ 371 Date: May 18, 2023

Related U.S. Application Data
(60) Provisional application No. 63/116,447, filed on Nov. 20, 2020.

(51) Int. Cl. C12N 5/071; C12M 3/06; C12M 1/32

(52) U.S. Cl. CPC C12N 5/0629; C12N 2513/00; C12M 23/12; C12M 23/16

(57) ABSTRACT

Disclosed are embodiments of a multicellular spheroid, including a core containing first skin cells; the core can have an average diameter of about 10 μm to about 900 μm. The multicellular spheroid may further include a shell overlaying the core, the shell having second skin cells. Further disclosed are systems containing such multicellular spheroids and methods of preparation and use of multicellular spheroids.

### FIELD

The present disclosure relates generally to organ-on-a-chip devices. More particularly the present disclosure relates to the utilization of microfluidic arrays used in an organ-on-a-chip device to produce spheroids containing skin cells. The spheroids can have a core-shell structure with the core containing first skin cells and the shell containing second skin cells so that the core-shell spheroids mimic skin tissues.

### BACKGROUND

Animal tests are intensively used in the traditional development of skincare products. However, experiments on animal models are expensive, time-consuming, and moreover, facing ethical criticism. In recent years, many legislative authorities around the world including the European Union, South Korea, and several states within the United States have officially banned animal tests for skincare products. Other countries like Canada are working on regulations to impart a similar ban on animal tests for skincare products. Therefore, an in vitro skin model is in great demand, and it would be very advantageous to find a rapid screening platform for producing large arrays of skin spheroids for high-throughput screening of multiple active ingredients (AIs) for skincare products in order to phase out animal testing.

Multicellular spheroids, that is, organotypic micrometer-size 3D cell aggregates, replicate many features of tissues and/or organs and thus have emerged as a promising in vitro model for drug screening, disease modelling, and developmental biology. Currently, spheroid-based skin models are not extensively used in research aimed at skincare. However, they are considered to be a promising model for screening AI candidates for skincare products. Given the advantages of the spheroid-based skin model, developing a robust skin spheroid model for AI screening would be highly beneficial for the use in both the cosmetic industry and research in the field of dermatology.

### SUMMARY

In some embodiments, disclosed herein is a multicellular spheroid, comprising a core comprising first skin cells, wherein the core has an average diameter of about 10 μm to about 900 μm and a shell overlaying the core, the shell comprising second skin cells. The first skin cells and the second skin cells, independently, may include dermis cells, epidermis cells and/or hypodermis cells. The dermis cells can include one or more of dermal fibroblasts, myofibroblasts, keloid fibroblasts, dermal papilla cells, sebocytes or dermal dendritic cells. The epidermis cells can include one or more of keratinocytes, epidermal stem cells, myoepithelial cells, melanocytes, Langerhans cells or Merkel cells. The hypodermis cells can include one or more of adipocytes, adipose derived stem cells, tenocytes, smooth muscle cells, gland stem cells or myoblasts. In certain embodiments, the first skin cells comprise human dermal fibroblasts and the second skin cells comprise keratinocytes. In some embodiments, the shell has an average thickness of about 1 μm to about 30 μm. The first skin cells may form an aggregate. In some embodiments, the first skin cells may be contracted. The shell may be adhered to the core. In one or more embodiments, the first skin cells are within a first hydrogel. The second skin cells may be within a second hydrogel.

According to one or more embodiments, further disclosed is a multicellular spheroid system, comprising a plurality of multicellular spheroids, each multicellular spheroid comprising a core comprising first skin cells, wherein the core has an average diameter of about 10 μm to about 900 μm and a shell overlaying the core, the shell comprising second skin cells, wherein the plurality of multicellular spheroids are arranged in an array of microwells. The array of microwells may be within a microfluidic device. Each microwell may have a cylindrical shape and/or a diameter of about 50 μm to about 1000 μm. The array of microwells may connect with a supplying channel. Each of the plurality of multicellular spheroids may have an average diameter that is within about ±1 μm to about ±10 μm of each other. In one or more embodiments, each of the plurality of multicellular spheroids has a spherical shape, a cylindrical shape or a disc shape.

In some embodiments, disclosed herein is a microfluidic-based process for preparing multicellular spheroids, comprising flowing a first skin cell-laden suspension into microwells and forming cell-laden droplets and inducing gelation to form gelled cell-laden droplets, supplying a first aqueous culture medium to the microwells to induce aggregation of the gelled cell-laden droplets and form compacted spheroids, flowing a second skin cell-laden suspension through the microwells to surround the compacted spheroids and induce gelation, and supplying a second aqueous culture medium to the microwells, wherein the second aqueous culture medium induces formation of the multicellular spheroids, each of the multicellular spheroids comprising a core comprising first skin cells and a shell comprising second skin cells. In some embodiments, one or more of the above-mentioned process steps is conducted at a temperature of about 37° C. under an atmosphere of about 5% CO₂. According to one or more embodiments, at least one of the first aqueous culture medium or the second aqueous culture medium flows from a preparation reservoir through microchannels to the microwells at a flow rate of about 0.001 mL/hr to about 0.5 mL/hr. In yet further embodiments, disclosed herein are multicellular spheroids comprising a core comprising first skin cells and a shell comprising second skin cells produced by this process.

In some embodiments, the first skin cell-laden suspension includes first skin cells suspended in an aqueous fluid, or in a hydrogel precursor solution. Preparing the first skin cell-laden suspension can include combining first skin cells with a first physiologically compatible hydrogel precursor solution. The first physiologically compatible hydrogel can include constituents that provide a biomimetic extracellular environment to assist in formation of the compact spheroids from the first cells and to support metabolic activity of the compact spheroids. In some embodiments, the first physiologically compatible hydrogel precursor solution includes about 0.3 wt % to about 2 wt %, or about 0.5 wt % aldehyde-functionalized cellulose nanocrystals (a-CNCs) and about 0.6 wt % to about 4 wt %, or about 1.5 wt % gelatin. The process can include flowing the first cell-laden suspension through microchannels into the microwells.

According to one or more embodiments, the first skin cell-laden suspension includes about 5×10³ cells/μL to about 5×10⁶ cells/μL, or about 4.9×10⁵ cells/μL dermal fibroblast cells. The second skin cell-laden suspension may have a cell/gel mass ratio of about 100:0 to about 50:50, or about 75:25. In some embodiments, the first skin cell-laden suspension flows from a preparation reservoir through microchannels to the microwells at a flow rate of about 0.01 mL/hr to about 0.30 mL/hr. In some embodiments, the second skin cell-laden suspension flows from a preparation reservoir through microchannels to the microwells at a flow rate of about 0.1 mL/hr to about 1 mL/hr.

In some embodiments, the second skin cell-laden suspension includes second skin cells suspended in an aqueous fluid or a hydrogel precursor solution. Preparing the second skin cell-laden suspension can include combining second skin cells with a second physiologically compatible hydrogel precursor solution. In some embodiments, the second physiologically compatible hydrogel includes constituents that provide a biomimetic extracellular environment to assist in formation of the shell containing the second skin cells surrounding the compact spheroids and to support metabolic activity of the shell and maintain structural integrity of the hybrid multicellular spheroids. The second physiologically compatible hydrogel precursor solution comprises about 0.3 wt % to about 2 wt %, or about 1.5 wt % aldehyde-functionalized cellulose nanocrystals (a-CNCs) and about 0.6 wt % to about 4 wt %, or about 3.0 wt % gelatin. The process may further include flowing the second skin cell-laden suspension through microchannels into the microwells.

In some embodiments, forming the cell-laden droplets includes a self-digitization process to fill each microwell with the first skin cell-laden suspension. The first aqueous culture medium may be conFIG.d to induce contraction of the cell-laden droplets to form the compacted spheroids. A surface of each of the compacted spheroids may include free space between the surface and walls of the microwells. In some embodiments, each of the microwells has a diameter of about 50 μm to about 1000 μm.

According to one or more embodiments, the process includes pre-wetting the microwells with a fluid immiscible to the first skin cell-laden suspension. After forming the gelled cell-laden droplets, the process may include flowing a fluid immiscible to the first skin cell-laden suspension through microchannels connected with the microwells for displacing any extra first skin cell-laden suspension located therein. After forming the compacted spheroids, the process may include flowing a fluid immiscible to the first aqueous culture medium through microchannels connected with the microwells for displacing any extra first aqueous culture medium located therein. After flowing the second skin cell-laden suspension through the microwells, the process may include flowing a fluid immiscible to the second skin cell-laden suspension through microchannels connected with the microwells for displacing any extra second skin cell-laden suspension located therein. In some embodiments, the immiscible fluid includes at least one of a biocompatible oil, an organic liquid, an organic oil or a fluorinated oil. The fluorinated oil may include about 0.1 wt % to about 1.0 wt %, or about 0.5 wt % of a fluorosurfactant.

A further understanding of the functional and advantageous aspects of the invention can be realized by reference to the following detailed description and drawings.

### BRIEF DESCRIPTION OF THE DRAWINGS

Embodiments of the present invention are described in greater detail with reference to the accompanying drawings.

FIG. 1 shows a schematic of the fabrication procedure of dermal fibroblasts spheroids (DFSs).

FIG. 2 shows a schematic of a procedure for preparing keratinocyte-dermal fibroblasts spheroids (KDFSs).

FIGS. 3A and 3B show the design of the microfluidic arrays in which FIG. 3A is a schematic of a quadruplet microfluidic array. Insert: Schematic of the fragment of the quadruplet microfluidic array with 100 μm wells—zoomed-in fragment of the quadruplet; Boom: Brigthfield image of a DFS in a microwell; and FIG. 3B is a real-size image of a spheroid-on-a-chip device containing 12 quadruplets.

FIGS. 4A to 4D show the 3D culture of human dermal fibroblasts (hDFs) in EKGels (i.e., a hydrogel made from a-CNC and gelation). FIGS. 4A and 4B show fluorescence images of calcein-AM- (green, live cells) and ethidium- (red, dead cells) stained hDFs cultured in EKGel with 0.5 wt % aldehyde-functionalized cellulose nanocrystals (a-CNC) and 1.5 wt % gelatin (FIG. 4A) and 1.5 wt % a-CNC and 3 wt % gelatin (FIG. 4B) on Day 5. FIG. 4C shows normalized metabolic activity measured by PrestoBlue assay of hDFs in hydrogels with varying a-CNC and gelatin concentrations. FIG. 4D shows collagen type I and fibronectin synthesis of hDFs in EKGels with different compositions on Day 5. Scale Bar is 100 μm. Sample Number=3. Two-way ANOVA tukey's post-hoc test: nsp>0.05, *p<0.05, **p<0.01, ***p<0.001

FIGS. 5A to 5C show characterization of DFSs formed on-chip. FIG. 5A shows fluorescence images of immunostaining on F-actin (red), E-cadherin (green), and nuclei (blue) of DFS formed in a 100 μm quadruplet. FIGS. 5B and 5C show fluorescence images of calcein-AM (green, live cells) and ethidium homodimer (red, dead cells) stained DFSs formed in 100 μm-diameter wells (FIG. 5B) and in 300 μm-diameter wells (FIG. 5C). Scale bar is 50 μm (FIG. 5A); 100 μm (FIG. 5B) and 300 μm (FIG. 5C).

FIGS. 6A to 6C show formation and maintenance of DFSs in a microfluidic platform. FIG. 6A are representative brightfield images of cell-laden droplets formed on Day 0 from the cell suspension with different cell density. FIG. 6B shows the success rate in forming well-defined cell-laden microgels on Day 0. FIG. 6C shows representative brightfield images of cell-laden microgels formed from the cell suspension with different cell density and maintained on Day 2. FIG. 6D shows the survival rate of cell-laden microgels as in FIG. 6C on Day 2. Scale bar is 100 μm. N=3.

FIGS. 7A to 7D show results of CFD simulations in a microfluidic array. FIG. 7A shows the circulation of flow in the microwell illustrated by velocity streamlines; FIG. 7B shows 3D contours and 3D surface arrows of velocity magnitude of flow around the spheroid; and FIGs. C and D show advection and diffusive flux of small molecule solute in the spheroid at two different concentrations (C=20 and 100 μg/m L).

FIGS. 8A to 8E show on-chip screening of vitamin C on DFSs. FIG. 8A shows collagen and fibronectin synthesis after a 24-hour (Day 3) treatment of vitamin C at different concentrations. FIGS. 8B and 8C show collagen type I and fibronectin synthesis after a 24-hour (Day 3) treatment and a 48-hour (Day 5) treatment of 0 μg/mL vitamin C (FIG. 8B) and 100 μg/mL vitamin C (FIG. 8C). FIG. 8D shows fluorescence images of immunostaining on collagen type I (red, Alexa 546), fibronectin (green, Alexa 488), and nuclei (blue, Hoechst 3442) of DFS on Day 5 (72 hours of vitamin C treatment) with 0 μg/mL and 100 μg/mL vitamin C. FIG. 8E shows the fluorescence intensity of collagen type I and fibronectin stained in the spheroids on Day 5 with 0 μg/mL and 100 μg/mL vitamin C. Scale bars are 50 μm.

FIGS. 9A and 9B show on-chip screening of retinol on DFSs. Collagen type I (FIG. 9A) and Fibronectin (FIG. 9B) secretion from DFSs after retinol treatment (Day 3: 24-hour treatment, Day 4: 48-hour treatment, Day 5: 72-hour treatment). nsp>0.05, *p<0.05, **p<0.01, ***p<0.001.

FIGS. 10A and 10B show the results of a 2D culture of human epidermal keratinocytes HaCaT on EKGels. FIG. 10A shows normalized metabolic activity measured by PrestoBlue assay of HaCaT on hydrogels with varying a-CNC and gelatin concentrations. FIG. 10B shows brightfield images of HaCaT seeded on EKGel on Day 1 and Day 5 with varying a-CNC and gelatin concentrations.

FIGS. 11A to 11E show characterizations of KDFSs for in vitro screening. FIG. 11A shows the structure of KDFSs. Keratinocytes were labelled with green 5-chloromethylfluorescein diacetate (CMFDA) and fibroblasts were labelled with red 4-({[4-(chloromethyl)phenyl]carbonyl}amino)-2-(1,2,2,4,8,10,10,11-octamethyl-10,11-dihydro-2H-pyrano[3,2-g:5,6-g′]diquinolin-1-ium-6-yl)benzoate (CMTPX). FIGS. 11B to 11D show immunofluorescent staining of involucrin and cytokeratin 14 (FIG. 11B), claudin 1 and fibronectin (FIG. 11C) and collagen type I and laminin 5 (FIG. 11D) in KDFSs. FIG. 11E shows collagen type I and Fibronectin secretion from KDFSs after 24-hour vitamin C treatment. nsp>0.05, *p<0.05, **p<0.01, ***p<0.001. The scale bar is 100 μm.

### DETAILED DESCRIPTION OF THE INVENTION

Generally speaking, the systems described herein are directed to the generation of massive arrays of core-shell skin spheroids. Various embodiments of the present invention are disclosed herein. The disclosed embodiments are exemplary, and it should be understood that the invention may be embodied in various and alternative forms. The FIGs. are not to scale and some features may be exaggerated or minimized to show details of particular elements while related elements may have been eliminated to prevent obscuring novel aspects. Therefore, specific structural and functional details disclosed herein are not to be interpreted as limiting but merely as a basis for the claims and as a representative basis for teaching one skilled in the art to variously employ the present invention. For purposes of teaching and not limitation, the illustrated embodiments are directed to processes for producing compact skin cell-containing spheroids, and core-shell skin spheroids using a self-digitization microfluidic device.

Reference throughout this specification to “one embodiment” or “an embodiment” means that a particular feature, structure, or characteristic described in connection with the embodiment is included in at least one embodiment. Thus, the appearances of the phrase “in one embodiment” or “in an embodiment” in various places throughout this specification are not necessarily all referring to the same embodiment. In addition, the term “or” is intended to mean an inclusive “or” rather than an exclusive “or.”

As used herein, the terms “about” or “approximately” in connection with a measured quantity refers to the normal variations in that measured quantity, as expected by one of ordinary skill in the art in making the measurement and exercising a level of care commensurate with the objective of measurement and the precision of the measuring equipment. In some embodiments, when “about” or “approximately” is used herein, this is intended to mean that the nominal value presented is precise within ±10%, such that “about 10” would include from 9 to 11. “About” or “approximately”, when used in conjunction with ranges of dimensions of microwells or cell density or other physical properties or characteristics of liquids or design of the microfluidic device, is meant to cover slight variations that may exist in the upper and lower limits of the ranges of dimensions so as to not exclude embodiments where on average most of the dimensions are satisfied but where statistically dimensions may exist outside this region. It is not the intention to exclude embodiments such as these from the present invention.

As used herein, the term “at least about” in connection with a measured quantity refers to the normal variations in the measured quantity, as expected by one of ordinary skill in the art in making the measurement and exercising a level of care commensurate with the objective of measurement and precisions of the measuring equipment and any quantities higher than that. The term “at least about” in connection with a measured quantity refers to the normal variations in the measured quantity, as expected by one of ordinary skill in the art in making the measurement and exercising a level of care commensurate with the objective of measurement and precisions of the measuring equipment and any quantities higher than that. In certain embodiments, the term “at least about” includes the recited number minus 10% and any quantity that is higher such that “at least about 10” would include 9 and anything greater than 9. This term can also be expressed as “about 10 or more.” Similarly, the term “less than about” typically includes the recited number plus 10% and any quantity that is lower such that “less than about 10” would include 11 and anything less than 11. This term can also be expressed as “about 10 or less.”

The term “and/or” as used in a phrase such as “A and/or B” herein is intended to include both “A and B” and “A or B”.

As used herein, the singular forms “a,” “an”, and “the” include plural references unless the context clearly indicates otherwise. Thus, for example, reference to “a precursor solution” includes a single precursor solution as well as to a mixture of two or more precursor solutions; and reference to a “reactant” includes a single reactant as well as a mixture of two or more reactants, and the like.

As used herein, the terms “comprises”, “comprising”, “including” and “includes” are to be construed as being inclusive and open ended, and not exclusive. Specifically, when used in this specification including claims, the terms “comprises”, “comprising”, “including” and “includes” and variations thereof mean the specified features, steps or components are included. These terms are not to be interpreted to exclude the presence of other features, steps or components.

As used herein, the terms “flow” or “flowing” (verb) is to be construed as being inclusive and open ended, and not exclusive. Specifically, when used in this specification including claims, the terms “flow” (verb) and variations thereof mean the fluids are flowed driven by a syringe pump. These terms are not to be interpreted to exclude the presence of other features, steps or components.

As used herein, the phrase “self-digitization” means an inherent fluidic phenomenon, in which an incoming aqueous sample divides itself into an array of microwells, each microwell having been primed with an immiscible phase. For example, it may refer to when a cell-laden suspension divides itself into the array of microwells; self-digitization is the mechanism for the droplet formation in the microwells.

As used herein ELISA is an acronym for “enzyme-linked immunosorbent assay” which is a plate-based assay technique designed for detecting and quantifying soluble substances such as peptides, proteins, antibodies, and hormones. Other names, such as enzyme immunoassay (EIA), are also used to describe the same technology.

The present invention according to embodiments herein provides an organ-on-a-chip device to grow massive arrays of skin spheroids for use in screening multiple active ingredients (AIs) for skincare products in a high-throughput manner. The skin spheroids-on-a-chip device is an effective and efficient solution to the problem of finding synthetic organ constructs to replace current animal testing models. Combining multicellular spheroids and microfluidic technology, fast growth of uniformly sized multicellular skin spheroids is achieved in a microfluidic array, allowing time-, cost-, and labor efficient screening of active ingredients. This platform can be further extended to effective screening of other substances such as pharmaceuticals or toxins.

Skin spheroids according to various embodiments herein preserve the physiological features of human skin. For example, several differentiation markers of epidermis were observed in epidermal spheroids even without the presence of an air-liquid interface, which is usually required in a traditional skin equivalent model. Major dermis extracellular matrix (ECM) proteins, e.g., fibronectin and collagen produced by dermal fibroblasts, were found in dermal spheroids. Thus, dermal spheroids preserve their inherent functions in producing and organizing the ECM of the dermis. Spheroid-based skin models offer simplicity in fabrication, the capability of scaling up, and the ability of time-effective AI screening. Skin spheroids may form within twenty four (24) hours from cell aggregates and become mature within 5 days. This fast fabrication of skin spheroids enables scaled-up production of massive in vitro skin models, which can accelerate the development of skincare products.

Strategies for spheroid formation and maintenance may have, at least, one of the following challenges: (i) broad distribution of spheroid dimensions (leading to uncertainty in AI screening); (ii) poorly controlled environments for spheroid growth; (iii) incompatibility with biological assays; and (iv) a limited throughput in AI screening, especially, for simultaneous screening of multiple AIs.

Microfluidics is the science and technology of systems that process or manipulate small (10⁻⁹ to 10⁻¹⁸ L) amounts of fluids using channels with dimensions of tens to hundreds of micrometers. Microfluidics offers the ability to improve the quality, functionality and high-throughput screening applications of current spheroid-based in vitro models. Existing microfluidics approaches to fabricate multicellular spheroids for in vitro screening can be classified into droplet-based and microchamber-based microfluidic devices. In the droplet-based microfluidics, multicellular spheroids are formed by generating droplets of high-density cell suspensions in hydrogel precursor solutions. This strategy enables high-throughput generation of uniformly sized spheroids in a biomimetic environment, however, it does not provide spatial control of each individual spheroid for observation. Furthermore, additional spheroid-trapping microfluidic devices are required to collect and store the spheroids for continuous long-term culture.

In microchamber-based microfluidics, the device contains an array of small chambers for spheroid growth and a common microchannel supplying the nutrition medium. Existing microchamber-based microfluidic devices generally does not favor the formation of cell-laden hydrogel droplets in the microchambers due to insufficient surface tension and thus cannot be used for growing spheroids in biomimetic microgels. In terms of the applications, limited work has been reported on skin spheroids in both droplet-based and microchamber-based microfluidic platforms. Furthermore, neither of these techniques has validated the use of skin spheroids for in vitro AI screening, as they lack the characterization of the skin spheroids' function to synthesize ECM proteins or their response to a benchmark AI.

In accordance with embodiments herein, one or more microfluidic devices that are capable of generating a large number of uniformly sized skin spheroids in biomimetic hydrogels have been developed. Such devices achieve spatial control over each individual spheroid and this platform is useful for in vitro screening of AIs.

One or more process as disclosed herein for production of spheroids utilize microfluidics for the production of core-shell skin spheroids comprising a core having one type of skin cell located therein and an outer shell having another type of skin cell thereby providing a biomimetic skin structure. The use of microfluidics as described herein enables the formation of uniform cell-laden droplets in microwells. Unique fluidic properties in the microfluidic channel may assist in the formation of such uniform cell-laden droplets. In micro-sized channels, surface forces dominate volumetric forces and the cell-laden droplets may be formed as a result of minimization of the interfacial energy between the oily phase and the aqueous phase in a fluid. The microfluidic device is also conFIG.d to provide a physiologically relevant dynamic fluidic environment to the spheroids. In the micro-sized channels, the fluid velocity surrounding the spheroids may be within a close-to-physiological range, mimicking the interstitial and luminal flow inside the human body. Further, the microfluidic device enables effective and efficient delivery of active ingredients to the spheroids. In the micro-sized channels, the two basic mechanisms of mass transport of biological molecules from flowing liquids, advection and diffusion, are recapitulated. The spheroids are able to uptake the active ingredients in a biomimetic way.

### Non-Compacted Spheroids

In some embodiments, disclosed herein are spheroids in a non-compacted form. For example, such spheroids may be cores for multicellular core-shell spheroids. The non-compacted spheroids also may be intermediates of a microfluidic-based process for preparing multicellular core-shell spheroids. In some embodiments, the non-compacted core structures may be formed by flowing a cell-laden suspension into microwells and forming cell-laden droplets. The cell-laden droplets may be formed by a self-digitization process.

The cell-laden suspension may include skin cells suspended in an aqueous fluid or a hydrogel precursor solution. Suitable skin cells include, but are not limited to, dermis cells, epidermis cells, hypodermis cells and combinations thereof. The dermis cells can include one or more of dermal fibroblasts, myofibroblasts, keloid fibroblasts, dermal papilla cells, sebocytes or dermal dendritic cells. The epidermis cells can include one or more of keratinocytes, epidermal stem cells, myoepithelial cells, melanocytes, Langerhans cells or Merkel cells. The hypodermis cells can include one or more of adipocytes, adipose derived stem cells, tenocytes, smooth muscle cells, gland stem cells or myoblasts.

After their formation, the cell-laden droplets may undergo gelation to form gelled cell-laden droplets. “Gelation” or “inducing gelation” as used herein refers to the formation of bonds between components that causes crosslinking; for example, it may be the formation of imine bonds between the a-CNC and gelatin, which causes crosslinking of the hydrogel precursor solution. The gelation may be conducted at a temperature of about 35° C. to about 45° C., or about 37° C., or any individual temperature or sub-range within this range.

Following gelation, an aqueous culture medium may be introduced into the microwells. The aqueous culture medium may be formulated to induce aggregation of skin cells within the gelled cell-laden droplets. Suitable aqueous culture mediums include, but are not limited to, Dulbecco's Modified Eagle Medium (DMEM), Dulbecco's Modified Eagle Medium/Nutrient Mixture F-12 (DMEM/F-12) and/or Minimum Essential Medium (MEM). In some embodiments, while the skin cells may aggregate to form a core, the skin cells may not compact resulting in a reduction in the size (e.g., average diameter) of the core. As such, the aggregated core may be a non-compacted core. In some embodiments, the aggregated, but non-compacted core, may be an intermediate structure (e.g., that is subsequently compacted) within a microfluidic-based process for preparing multicellular core-shell spheroids. In some embodiments, the non-compacted core structures may be overlaid by a shell composition that also comprises skin cells. In some embodiments, the skin cells in the shell are different from the skin cells in the core.

In one or more embodiments, a microfluidic-based process may be used to produce skin cell containing spheroids in a microfluidic array containing an array of microwells. In some embodiments, the process comprises preparing an aqueous suspension of skin cells, for example, in an aqueous fluid and/or in a physiologically compatible hydrogel precursor solution, to produce a skin cell-laden suspension. The suspension may flow through microchannels into associated microwells of a microfluidic array. In some embodiments, the microwells may be pre-wetted with a fluid immiscible to the aqueous suspension to form cell-laden droplets through self-digitization. For example, the fluid immiscible to the aqueous suspension may flow through the microchannels to displace any extra cell-laden suspension located in the microchannels. The flow of the fluid immiscible to the aqueous suspension may cause formation of the skin cell-laden droplets within the microwells.

A culture medium may be introduced into the microwells to displace the immiscible fluid with a culture medium that is conFIG.d to induce aggregation of the gelled droplets to form non-compacted spheroids or intermediates. The liquid that is immiscible to the aqueous suspensions and the aqueous culture media may be a biocompatible oil, an organic liquid, an organic oil, a fluorinated oil and/or combinations thereof. In some embodiments, the immiscible fluid is a fluorinated oil containing a fluorosurfactant in an amount about 0.1 wt % to about 1.0 wt %, or about 0.2 wt % to about 0.5 wt %, or any individual value or sub-range within these ranges.

Non-compacted skin cell spheroids as described above can form a platform for core-shell spheroid structures and/or for various types of skin compatibility testing.

### Core-Shell Spheroids

Core-shell spheroids according to embodiments herein may be useful to produce a system that mimics human skin. In some embodiments, a multicellular spheroid includes a core containing first skin cells. Suitable skin cells include, but are not limited to, dermis cells, epidermis cells, hypodermis cells and combinations thereof. The dermis cells can include one or more of dermal fibroblasts, myofibroblasts, keloid fibroblasts, dermal papilla cells, sebocytes or dermal dendritic cells. The epidermis cells can include one or more of keratinocytes, epidermal stem cells, myoepithelial cells, melanocytes, Langerhans cells or Merkel cells. The hypodermis cells can include one or more of adipocytes, adipose derived stem cells, tenocytes, smooth muscle cells, gland stem cells or myoblasts.

The core may have a spheroidal, cylindrical or disc shape. The aggregation and/or compaction of skin cells may not form a perfect sphere, cylinder or disc and thus the resulting aggregate and/or compact may be defined by its average diameter, average length and/or average height. According to various embodiments, the core may have an average diameter of about 10 μm to about 900 μm, about 10 μm to about 500 μm, about 10 μm to about 300 μm, about 10 μm to about 200 μm, about 10 μm to about 100 μm, or any individual average diameter or sub-range within these ranges. In some embodiments, the core may have an average diameter of at least about 10 μm. Without being bound by any particular theory, it is believed that a diameter of at least about 10 μm may provide a size or a sufficient number of skin cells on which a shell layer may be formed.

Core-shell spheroids according to embodiments herein also include a shell overlaying the core. The shell may contain skin cells and may be adhered to the core. The skin cells in the shell may be the same or different than the skin cells in the core. Suitable skin cells for the shell include, but are not limited to, dermis cells, epidermis cells, hypodermis cells and combinations thereof. The dermis cells can include one or more of dermal fibroblasts, myofibroblasts, keloid fibroblasts, dermal papilla cells, sebocytes or dermal dendritic cells. The epidermis cells can include one or more of keratinocytes, epidermal stem cells, myoepithelial cells, melanocytes, Langerhans cells or Merkel cells. The hypodermis cells can include one or more of adipocytes, adipose derived stem cells, tenocytes, smooth muscle cells, gland stem cells or myoblasts. In certain embodiments, the core can include one or more types of skin cells found in the dermis layer; the shell can include one or more types of skin cells found in the epidermis layer or the hypodermis layer. In a particular embodiment, the core contains human dermal fibroblasts and the shell contains keratinocytes.

According to embodiments, the skin cells in the shell may not maintain the same thickness around the core thus the resulting shell may be defined by its average thickness. The shell may have an average thickness of about 1 μm to about 30 μm, about 2 μm to about 25 μm, or any individual thickness or sub-range within these ranges.

According to some embodiments, the skin cells in the core are contracted forming the core such that the contracted core has an average diameter that is smaller than the aggregate of skin cells formed prior to contraction. For example, contraction of the core may reduce the average diameter of the aggregated core by about 0.1 μm to about 15 μm, or any individual value or sub-range within this range.

In some embodiments, the skin cells in the core are within a hydrogel. The hydrogel may be a physiologically compatible hydrogel precursor solution. In some embodiments, the physiologically compatible hydrogel includes constituents that provide a biomimetic extracellular environment to assist in formation of the compact spheroids from the skin cells in the core and to support metabolic activity of the compact spheroids. In some embodiments, the physiologically compatible hydrogel precursor solution includes aldehyde-functionalized cellulose nanocrystals (a-CNCs) in an amount of about 0.3 wt % to about 2 wt %, or about 0.5 wt %, or any individual value or sub-range within this range. Additionally, or alternatively, the hydrogel precursor solution may include gelatin in an amount of about 0.6 wt % to about 4 wt %, or about 1.5 wt %, or any individual value or sub-range within this range.

In some embodiments, the skin cells within the shell are in a hydrogel. The hydrogel may be a physiologically compatible hydrogel precursor solution. The physiologically compatible hydrogel may include constituents that provide a biomimetic extracellular environment to assist in formation of the shell containing the skin cells surrounding the compact spheroids and to support metabolic activity of the shell and maintain structural integrity of the hybrid multicellular spheroids. In some embodiments, the physiologically compatible hydrogel precursor solution includes aldehyde-functionalized cellulose nanocrystals (a-CNCs) in an amount of about 0.3 wt % to about 2 wt %, or about 1.5 wt %, or any individual value or sub-range within this range. Additionally or alternatively, the hydrogel precursor solution may include gelatin in an amount of about 0.6 wt % to about 4 wt %, or about 3.0 wt %, or any individual value or sub-range within this range.

In some embodiments, disclosed herein are core-shell spheroids formed by a microfluidic-based process. The core may be comprised of one type of skin cells (e.g., human skin cells found in the dermis layer) and the shell may be comprised of a second type of skin cells (e.g., human skin cells found in the epidermis layer or the hypodermis layer). The core-shell spheroids may be formed within a microfluidic array device containing an array of microwells connected to a supplying channel.

The core-shell spheroids may be formed by a process that includes suspending first skin cells in a first physiologically compatible hydrogel precursor solution to produce an aqueous first skin cell-laden hydrogel precursor solution, and flowing this first cell-laden hydrogel precursor solution through microchannels into associated microwells of a microfluidic array to form first cell-laden droplets located inside the microwells. Inside the microwells, the first skin cell-laden droplets undergo gelation. After gelation, a first aqueous culture medium is flowed to the microwells and this first culture medium is selected to induce aggregation and contraction of the gelled cell-laden droplets to form compacted spheroids. These compacted spheroids have a volume that is smaller than the volume of the microwells such to provide free space around the compacted spheroids in the microwells so that more liquid can flow into the microwells containing the compacted spheroids. Second skin cells are suspended in a second physiologically compatible hydrogel precursor solution which is then flowed through microchannels into the microwells to engulf the compacted spheroids, whereupon these second skin cell-laden hydrogel precursor solution engulf the compacted spheroids by filling in the available volume in each microwell where the precursor solutions undergo gelation. After gelation of the second skin cell-laden hydrogel engulfing the compacted spheroids, a second culture medium is flowed to the microwells wherein this second culture medium is selected to induce formation of hybrid multicellular spheroids with a core containing first skin cell and a shell containing the second skin cells.

Prior to flowing the cell-laden suspension of first skin cells, the microwells may be wetted with a fluid immiscible to the first skin cell-laden suspension. Suitable immiscible fluids include, but are not limited to, a biocompatible oil, an organic liquid, an organic oil and/or combinations thereof. In some embodiments, the immiscible fluid is a fluorinated oil or containing a fluorosurfactant in an amount of about 0.1 wt % to about 1.0 wt %, about 0.5 wt %, or any individual value or sub-range within this range.

After formation of the first cell-laden droplets, the process may include flowing immiscible fluid through the microchannels for displacing any extra first cell-laden hydrogel precursor solution located in the microchannels. Similarly, after formation of the compacted spheroids the process may include flowing immiscible fluid flowing through the microchannels for displacing any extra first aqueous culture medium located therein. Further, after flowing the second aqueous suspension of second skin cells to engulf the compacted spheroids, the process may include flowing immiscible fluid through the microchannels for displacing any extra aqueous suspension of second skin cells located therein.

The hydrogel precursor solution is preferably freshly prepared before the loading of the cells. Once the cores are produced, the outer shell structure is preferably flowed into the microwells in a time range of from about 12 to about 48-hours after the gelation of the first cell-laden droplets.

Each of the core and shell can include any combination of skin cells as described hereinabove. In addition, for some embodiments, it will be appreciated that the core may contain more than one type of skin cell found in the layer in the dermis skin layer, and the outer shell layer may contain more than one type of skin cell typically found in epidermis or hypodermis skin layer. For example, dermal fibroblasts and dermal papilla cells can be used to produce the core and epidermal keratinocytes and melanocytes can be mixed and delivered as the second cell-laden hydrogel precursor solution to be the shell. Further, skin cells normally only found in the outer skin layer may be used in the core and vice versa skin cells normally found in the skin beneath the epidermis may be included in the outer shell skin layer, depending on the types of studies being undertaken.

The first physiologically compatible hydrogel is comprised of constituents selected on the basis that they provide biomimetic extracellular environment to assist in formation of the compact spheroids from the first cells and to support metabolic activity of the compacted spheroids. Similarly, the second physiologically compatible hydrogel is comprised of constituents that provide biomimetic extracellular environment to assist in formation of the shell containing the second skin cells surrounding the compacted spheroids and to support metabolic activity of the shell and maintain structural integrity of the compact spheroids.

The steps of preparing the suspensions and flowing them to the microwells may be conducted at about room temperature under an atmosphere level of carbon dioxide (CO₂). During incubation when supplying the aqueous culture mediums to the microwells, the process may be conducted at a temperature of about 35° C. to about 45° C., greater than about 35° C. to about 45° C., about 37° C., or any individual temperature of sub-range within these ranges, and under an atmosphere CO₂ in a concentration of about 1% to about 10%, about 5%, or any individual concentration or sub-range within this range.

In some embodiments, the microwells have a diameter in a range of about 50 μm to about 1000 μm, or any individual value or sub-range within this range. The diameter of each microwell may depend on the type of skin cells being used. The diameter may be sufficiently large enough to accommodate enough cells to form multicellular spheroids. The diameter may be less than a reasonable value so that nutrient can be effectively transported deep into the core of the spheroids and waste can be effectively exchanged out of the spheroids.

### Core Containing Dermal Fibroblasts and Shell Containing Keratinocytes Cells

When the first skin cells are human dermal fibroblasts, and the second skin cells are human keratinocytes cells, the constituents of the first physiologically compatible hydrogel precursor solution comprises about 0.3 to about 2 wt % aldehyde-functionalized cellulose nanocrystals (a-CNCs) and about 0.6 to about 4 wt % gelatin. A preferred composition of the first physiologically compatible hydrogel precursor solution comprises about 0.5 wt % aldehyde-functionalized cellulose nanocrystals (a-CNCs) and about 1.5 wt % gelatin.

The concentration of the dermal fibroblast cells in the precursor solution can have a density in a range from about 5×10³ to 5×10⁶ cells/μL, while a preferred concentration of the dermal fibroblast cells in the precursor solution has a density of about 4.9×10⁵ cells/μL.

The second physiologically compatible hydrogel precursor solution comprises about 0.3 to about 2 wt % aldehyde-functionalized cellulose nanocrystals (a-CNCs) and about 0.6 to about 4 wt % gelatin. A preferred composition of the second physiologically compatible hydrogel precursor solution comprises about 1.5 wt % a-CNCs and about 3.0 wt % gelatin. The second hydrogel precursor solution can have a cell/gel mass ratio in a range from about 100:0 to about 50:50. A preferred cell/gel mass ratio is about 75:25 for epidermal keratinocytes.

The microwells have a diameter in a range from about 50 μm to about 1000 μm. The microwells within such a range provide sufficient space to accommodate the first and second skin cells to form core-shell spheroids, ensure the effective nutrient and waste transport across the spheroids, and prevent the formation of necrotic core.

The first skin cell-laden hydrogel precursor solution is flowed from a preparation reservoir through the microchannels to the microwells at a flow rate in a range from about 0.01 mL/hr to about 0.30 mL/hr. The second skin cell-laden hydrogel precursor solution is flowed from a preparation reservoir through the microchannels to the microwells at a flow rate in a range from about 0.1 mL/hr to about 1 mL/hr. The culture medium is flowed from a preparation reservoir through the microchannels to the microwells at a flow rate in a range from about 0.001 mL/hr to about 0.5 mL/hr.

The present disclosure will now be illustrated using the non-limiting examples of inner core skin cells being dermal fibroblasts and the skin cells incorporated into the outer skin layer being keratinocytes. More particularly, the present is illustrated by a process of producing spheroids-based skin models containing only dermal fibroblasts spheroids or core-shell skin spheroids containing dermal fibroblasts cores and epidermal keratinocytes outer layers in a microfluidic array. In this example, dermal fibroblasts spheroid (DFS) is a multicellular spheroid formed by aggregation of dermal fibroblasts, representing human dermal tissue, and a keratinocytes-dermal fibroblasts spheroid (KDFS) is a core-shell hybrid spheroids form by a core of DFSs and a shell of keratinocytes, representing epidermal tissue and dermal tissue of human skin.

### Multicellular Spheroid System

Further described herein are one or more embodiments of a multicellular spheroid system. Multicellular spheroid systems as described herein may include a plurality of multicellular spheroids according to one or more embodiments described herein. In some embodiments, each multicellular spheroid includes a core containing first skin cells. The core may have an average diameter of about 10 μm to about 900 μm, or any individual value or sub-range within this range. The shell may overlay the core and may have a thickness of about 1 μm to about 30 μm, or any individual value or sub-range within this range. The shell may include second skin cells. In some embodiments, the first skin cells are different from the second skin cells.

The plurality of multicellular spheroids within the multicellular spheroid system may be arranged within an array of microwells. In some embodiments, the array of microwells is within a microfluidic device as described herein. Each microwell may have a cylindrical shape although other shapes are contemplated including spherical, square, rectangular or any suitable shape for forming spheroids as described herein. According to one or more embodiments, each microwell has a diameter of about 50 μm to about 1000 μm, or any individual diameter or sub-range within this range. In some embodiments, each of the plurality of multicellular spheroids has an average diameter that is within about ±1 μm to about ±10 μm, or any individual value or sub-range within this range, of each other.

In one or more embodiments, the system may include a supplying channel connected to the array of microwells. The supplying channel may be conFIG.d to enable self-digitization of fluids flowing through the channel and into the microwells connected thereto.

### Experimental Section

### Preparation of EKGel

The EKGel precursor solution was prepared by mixing an aqueous suspension of aldehyde-functionalized cellulose nanocrystals (a-CNCs) and a solution of gelatin in 10 wt % in DMEM complete culture medium. The a-CNCs were prepared by introducing aldehyde groups to the surface of cellulose nanocrystals (CNC) via oxidation with sodium periodate (NalO₄). NalO₄ was added to the suspension of CNCs at a weight ratio from about 0.1:1 to 10:1 (NalO₄:CNC). The mixture was stirred at room temperature. After about 1 hr to 48 hr, the reaction was quenched. The suspension of aldehyde-modified CNCs (a-CNCs) was dialyzed against deionized water.

An aqueous suspension of a-CNCs with concentration of 2.7-3.7 wt % was diluted with a 10×HBSS buffer. This a-CNC suspension in a 1×HBSS is referred to as the stock a-CNC suspension. In the present example, two different compositions of EKGel were prepared for the culture of two different skin cells, hDFs and human epidermal keratinocyte cell line, HaCaT. EKGel I contained 0.5 wt % a-CNCs and 1.5 wt % gelatin and was used for culture of hDFs; EKGel II contained 1.5 wt % a-CNCs and 3.0 wt % gelatin and was used for culture of keratinocytes. Prior to mixing, the stock a-CNC suspension and gelatin solution were subjected to 30 min ultraviolet (UV) light irradiation for sterilization. The EKGel precursor solution was then mixed with cell suspension to reach a particular cell density (as specified in Generation and growth of DFSs/KDFSs in the microfluidic device). The cell-laden EKGel precursor solution was then loaded into the microfluidic device. The cell-laden EKGel precursor solution loaded in the microfluidic device was incubated for two hours at 37° C. in the incubator with a constant 5% CO₂ supply.

### Cell Culture

Primary hDFs were purchased from ATCC® (PCS-201-010™) and the human keratinocyte cell line, HaCaT, was purchased from CLS Cell Lines Service GmbH (Germany). Both types of cells were cultured in T75 tissue culture flasks with 10 mL complete cell culture medium in the incubator at 37° C. with a constant 5% CO₂ supply. The complete cell culture medium consisted of DMEM, 10% (v/v) fetal bovine serum and 1% (v/v) penicillin/streptomycin. At approximately 90% confluency, the cells were passaged. The hDFs were first, detached from T75 tissue culture flasks using 5 min incubation in 3 mL of 0.25% trypsin-EDTA solution. Keratinocytes were incubated with 3 mL 0.25% trypsin-EDTA solution for 10 min. Subsequently, 5 mL of complete culture medium was added to stop trypsinization. The cell suspension was centrifuged at 184 g for 3 min. The resulting cell pellet was then re-suspended in 1000 μL of medium and 300 μL of the cell suspension was transferred into a new flask. In this work, hDFs at Passage 5-11 were used; keratinocytes at Passage 35-50 were used.

### Fabrication of Microfluidic Devices

A microfluidic device was fabricated using a standard soft lithography process. Polydimethylsiloxane (PDMS) (an elastomer base and a curing agent mixed at a weight ratio of 10:1) was cast onto the silicon wafer master prepared by the photolithography process. A 75 mm×50 mm glass slide was spin-coated with 1 mL of PDMS at 800 g for 15 seconds. The PDMS on the master and the glass slide was cured for 2 hours in an oven a 75° C. The cured PDMS with the microchannel features was off-peeled from the master. The off-peeled PDMS and PDMS-coated glass slides were subjected to air plasma for 90 sec and subsequently, bonded together. The assembled devices were placed in a 115° C. oven overnight to recover the hydrophobicity.

### Generation and Growth of DFSs/KDFSs in the Microfluidic Device

The microfluidic device was first, purged for 20 min with fluorinated oil with 0.5 wt % 008-fluorosurfactant (RAN Biotechnologies, USA) at a flow rate of 0.1 mL/hr. Subsequently, the supplying channel and the microwells were filled with the hDFs-in-EKGel I suspension with a cell density of 4.9×10⁵ cells/μL. After that, the cell suspension in the supplying channel was replaced with fluorinated oil with 0.5 wt % surfactants. The inlet and outlet of the microfluidics quadruplet device were then sealed with PCR sealing tape and incubated at 37° C. for 2 hours, allowing EKGel to gel. Subsequently, the complete culture medium was continuously flowed through the supplying channel at the flow rate of 0.025 mL/hr. On the second day (Day 1), hDFs-laden EKGel droplets transformed into dermal fibroblasts spheroids (DFSs). Due to the contraction of DFSs, free space was present in each microwell, which allowed the incorporation of a second layer. To introduce another layer of keratinocytes, the culture medium was replaced by fluorinated oil with 0.5 wt % surfactants.

Keratinocytes were suspended in EKGel II precursor solution at a cell/gel mass ratio of 75:25. The keratinocytes-in-EKGel II suspension was then purged into the channel and filled the microwells. Consequently, the cell suspension in the supplying channel was washed away by fluorinated oil mixed with 0.5 wt % surfactants. The device was sealed and incubated at 37° C. for 1 hour. Finally, the complete culture medium was flowed to the microfluidic device at the flow rate of 0.025 mL/hr. On Day 2, keratinocytes aggregated and integrated with the hDF core to form layered skin spheroids, KDFSs.

To study the effect of vitamin C or retinol on DFSs or KDFSs, on Day 2 the complete culture medium was replaced with 1% FBS DMEM culture medium with 1% (v/v) penicillin/streptomycin containing 0, 20, or 100 μg/mL vitamin C. Retinol at concentrations of 0, 30, 45, 60 μM was dissolved in 1% FBS DMEM culture medium with 1% (v/v) penicillin/streptomycin and 0.1% dimethyl sulfoxide (DMSO). The culture supernatant was collected from the outlet every 24-hours for analysis.

### Structure Analysis of KDFSs

To study the structure of KDFSs, both hDFs and keratinocytes were labelled with two different cell trackers, Green CmicrofluidicsDA Dye (Invitrogen™) and Red CMTPX Dye(Invitrogen™), respectively. To stain hDFs, Red CMTPX Dye was first, dissolved in pure DMSO and then diluted with a serum-free DMEM medium to a final concentration of 15 μM as the hDFs staining solution. To stain keratinocytes, Green CmicrofluidicsDA Dye was first dissolved in pure DMSO and then diluted with a serum-free DMEM medium to a final concentration of 20 μM as the keratinocytes staining solution. hDFs or keratinocytes in T75 tissue culture flasks were then incubated with their corresponding staining solution for 30 min. The stained cells were then detached from tissue culture flasks for microfluidics experiments.

### Immunofluorescence Staining of DFSs/KDFSs on the Chip

The immunofluorescence staining on DFSs/KDFSs formed in the microfluidic device was conducted by flowing a series of solutions at a flow rate of 0.15 mL/hr into the supplying microchannel in the following order. First, 1× Hanks' Balanced Salt Solution (HBSS) was flowed into the device for 20 min to remove the cell culture medium. Subsequently, a 5% formalin solution in HBSS was flowed to fix the DFSs/DKFSs. Subsequently, the formalin was washed away by purging 0.1 M glycine in 1×HBSS for 60 min. To permeate the DFSs/KDFSs, 0.5% triton-X in 1×HBSS was flowed into the device for 30 min. Subsequently, an immunofluorescence (IF) solution (consisting of 0.05 wt % NaN₃, 0.1 wt % bovine serum albumin, 0.2 vol. % triton-X-100 and 0.05 vol. % Tween-20 in HBSS) was flowed to the microfluidics for 45 min. Primary blocking solution (10 wt % goat serum in IF washing solution) was then flowed for 60 min. Primary antibodies (in primary blocking solution) were then flowed into the device for another 60 min to replace the blocking solution. The device was incubated overnight at 4° C. On the second day, Hoechst 33342 solution was flowed into the channel for 60 min to stain the cell nuclei. Secondary antibodies were added to the Hoechst solution and flowed into the device together. Details about the antibodies were listed in Table 1.

TABLE 1
Antibodies used for immunofluorescence staining

| | | Secondary |
|---|---|---|
| Antigen | Primary Antibody | Antibody |
| E-cadherin | Alexa Fluor 488 E-Cadherin rabbit monoclonal | |
| | antibody (1:800 dilution, Cell Signaling | |
| | Technology ™) | |
| F-actin | 165 nM/mL Alexa Fluor 568 Phalloidin (Life | |
| | technologies ™) | |
| Collagen | Rabbit anti-human collagen | Goat anti- |
| Type I | type I polyclonal antibody | rabbit IgG |
| | (1:250 dilution, Invitrogen ™) | Alexa Fluor |
| Claudin 1 | Rabbit anti-human claudin 1 | 546 (1:500 |
| | polyclonal antibody (1:100 | dilution, |
| | dilution, Invitrogen ™) | Invitrogen ™) |
| Involucrin | Rabbit anti-human involucrin | |
| | polyclonal antibody (1:100 | |
| | dilution, Invitrogen ™) | |
| Fibronectin | Mouse anti-human fibronectin | Goat anti- |
| | monoclonal antibody (1:100 | mouse IgG |
| | dilution, Invitrogen ™) | Alexa Fluor |
| Cytokeratin | Mouse anti-human cytokeratin | 488 (1:400 |
| 14 | 14 monoclonal antibody (1:100 | dilution, |
| | dilution, Invitrogen ™) | Invitrogen ™) |
| Laminin 5 | Mouse anti-human laminin 5 | |
| | monoclonal antibody (1:100 | |
| | dilution, Chemicon ®) | |

| | |
|---|---|
| Nuclei | 0.5 ng/mL Hoechst 33342 solution (Invitrogen ™) |

### Enzyme-Linked Immunosorbent Assay (ELISA)

The amount of procollagen type I and fibronectin synthesized by hDFs and released in the supernatant were quantified using two ELISA kits, namely, Procollagen Type I C-Peptide (PIP) ELISA kit (MK101, Takara Bio Inc.) and Fibronectin ELISA kit (MK115, Takara Bio Inc.). Procollagen type I was used as the indicator for the collagen type I synthesized from hDFs. To determine the amount of procollagen type I synthesized by the cells, 20 μL collected culture supernatant and 100 μL of the anti-PIP antibody-peroxidase (POD) were added into a 96-well microplate coated with anti-PIP antibody and incubated at 37° C. for 3 hours. To determine the amount of fibronectin synthesized by the cells, 100 μL of the supernatant was first, added to the antibody-coated 96-well microplate for 1 hour at 37° C., thus allowing the fibronectin in the supernatant to bond to the antibody-coated on the microplate.

The supernatant was then discarded, and the microplates were washed with 1×PBS (MK021, Takara Bio Inc.) three times. Subsequently, the captured fibronectin was tagged by adding 100 μL of anti-human fibronectin antibody-POD conjugate into the microplate and incubating it for another 1 hour at 37° C. Upon completion of the antibody-POD conjugate incubation for both assays, the liquid content in the microwells was discarded and the microwells were washed four times with 1×PBS. 100 μL substrate solution (solution of 3,3′,5,5′-tetramethylbenzidine) was then added to the wells and incubated at room temperature for 15 min. Subsequently, 100 μL stop solution (MK021, Takara Bio Inc.) was added to each well. After stopping the reaction between POD and substrate, the absorbance of the contents of each microwell at 450 nm was measured by the CLARIOstar plate reader.

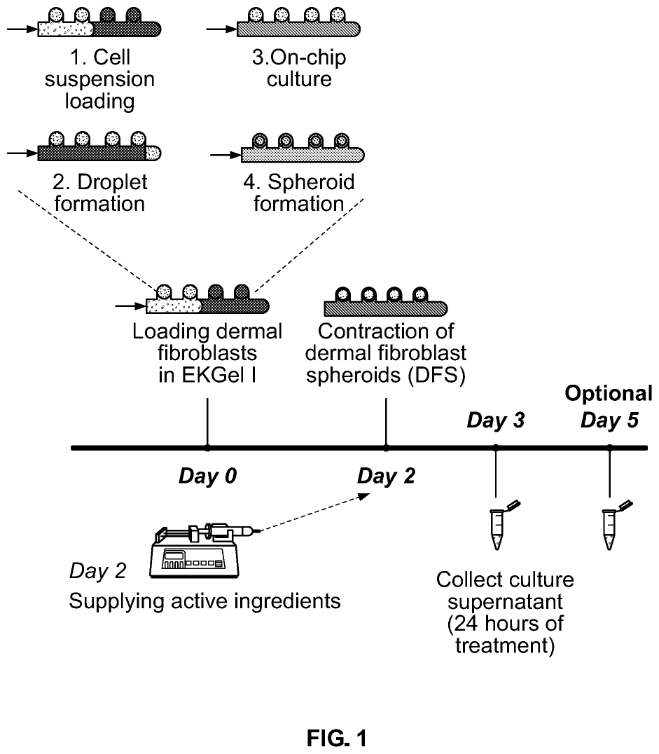

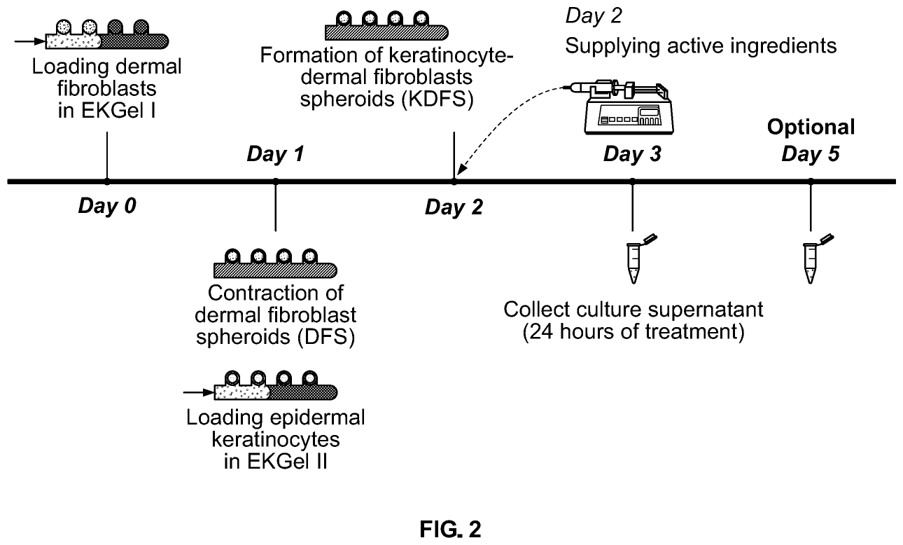

The fabrication involves several steps as shown in FIG. 1. For DFSs production, the procedure is illustrated in FIG. 1: 1) fibroblasts are suspended in an EKGel I (0.5 wt. % aldehyde-functionalized cellulose nanocrystals (a-CNC) and 1.5 wt. % gelatin) precursor solution at 4.9×10⁵ cells/mL—EKGel I is a hydrogel made from a-CNC and gelation; 2) fibroblasts-laden EKGel I precursor solution is flowed into the microfluidic array to form droplets inside the microwells; 3) culture medium is flowed to the channel after gelation of fibroblasts-laden EKGel I; 4) on the second day, fibroblasts aggregate and contract, forming compacted spheroids; 5) on the third day, DFSs are ready for screening. For KDFSs, the first four steps are identical and additional steps are required from Step 5 (FIG. 2): 5) keratinocytes are suspended in EKGel II (1.5 wt. % a-CNC and 3 wt. % gelatin) precursor solution at a cell/gel mass ratio of 75:25; 6) keratinocytes-laden EKGel II precursor solution is flowed into the microfluidic array to engulf the fibroblasts spheroids; 7) culture medium is flowed to the channel after gelation of keratinocytes-laden EKGel 8) on the third day, hybrid spheroids are formed and ready for screening.

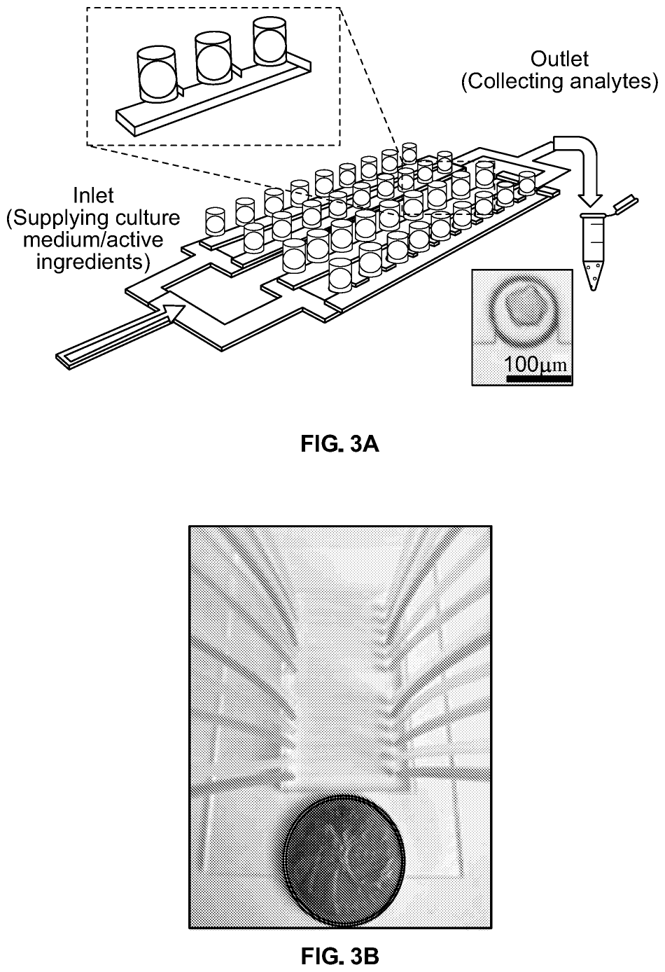

The design of the microfluidic device is illustrated in FIGS. 3A and 3B. The device contains four parallel rows of microwells, which have a common inlet and outlet (FIG. 3A). Later in the text, we refer to this microfluidic device as a “quadruplet”. Each row comprises 50 cylinder-shaped microwells for DFS growth; thus potentially, 200 spheroids can be formed per quadruplet. The culture medium or the AI candidates are flowed from the common inlet and the culture supernatant is collected from the common outlet to analyze the protein synthesized. FIG. 3B shows the real-size image of the spheroid-on-a-chip device containing 12 quadruplets.

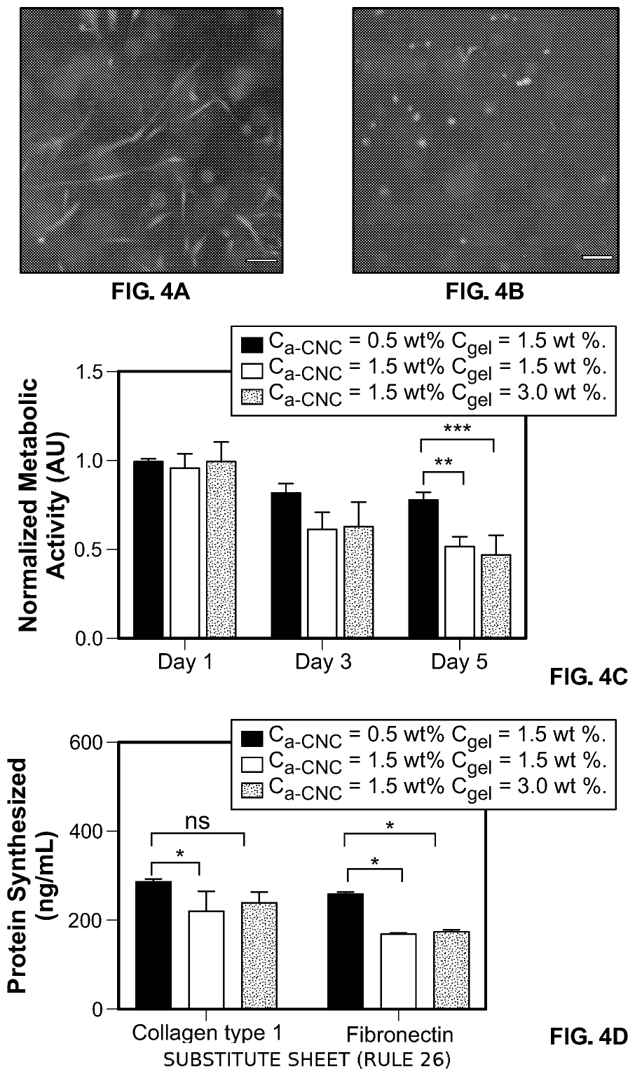

To optimize the EKGel I composition for the growth of fibroblasts, hDFs were encapsulated in different EKGel compositions (FIGS. 4A to 4D). At the concentration of a-CNCs, C_{a-CNC}, of 0.5 wt % the hDFs had a spindle-type morphology and spread out (FIG. 4A), while at C_{a-CNC}=1.5 wt % the hDFs acquired a spherical morphology and exhibited reduced spreading (FIG. 4B). The spreading of hDFs indicated that EKGel had an appropriate stiffness and available cell attachment ligands for hDFs to recapitulate their morphology in human dermis. Meanwhile, the metabolic activity of hDFs remained highest in EKGel with 0.5 wt % a-CNC and 1.5 wt % gelatin (FIG. 4C). To evaluate the functionality of hDFs encapsulated in the EKGel scaffold, we examined the synthesis of collagen type I and fibronectin via ELISA. The highest amount of collagen type I and fibronectin synthesized was achieved when the hDFs were encapsulated in EKGel with 0.5 wt % a-CNC and 1.5 wt % gelatin (FIG. 4D). Taken together, in EKGel with 0.5 wt % a-CNC and 1.5 wt % gelatin, hDFs preserved a characteristic spindle morphology, maintained the highest metabolic activities, and produced the most dermal ECM proteins.

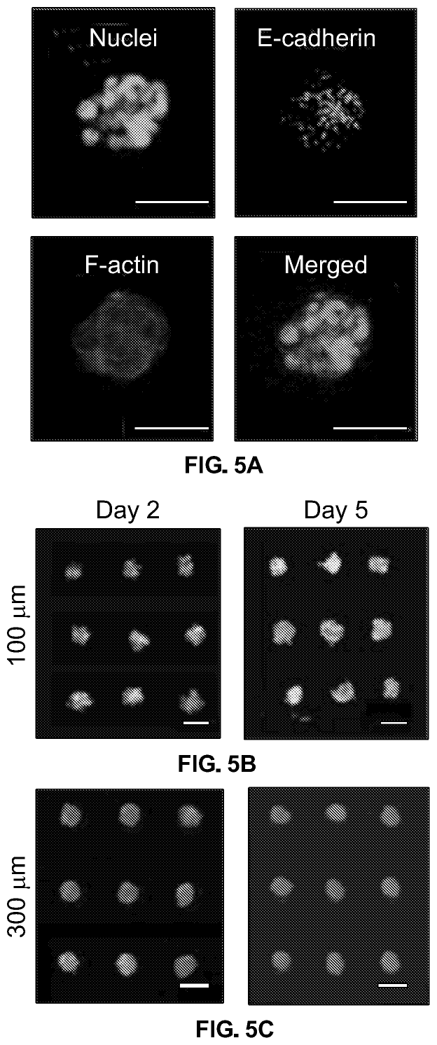

To characterize DFSs, the expression of cell-cell adhesion junctions (i.e. E-cadherin) and cytoskeleton (i.e. F-actin) was examined via immunofluorescent staining (FIG. 5A). The positive staining of E-cadherin confirmed the formation of multicellular spheroids. F-actin staining demonstrated the elongated cytoskeleton network of dermal fibroblasts in DFSs. To evaluate the viability of hDFs during on-chip culture, live/dead staining was conducted on Days 2 and 5. FIGS. 5B and C show fluorescence microscopy images of hDFs stained with ethidium homodimer for dead cells (red) and calcein-AM for live cells (green). In 100 μm-diameter microwells, a major fraction of cells remained alive over 5 days (FIG. 5B). In contrast, in 300 μm-diameter microwells, a necrotic core emerged in the DFSs on Day 2, which became particularly clear on Day 5 (FIG. 5C). We attribute this effect to the poor nutrient and oxygen transport from the medium to the core of 300 μm-diameter DFSs, in comparison with 100 μm-diameter DFSs. Therefore, DFSs encapsulated in microgels with dimensions smaller than 100 μm had higher viability than larger DFSs.

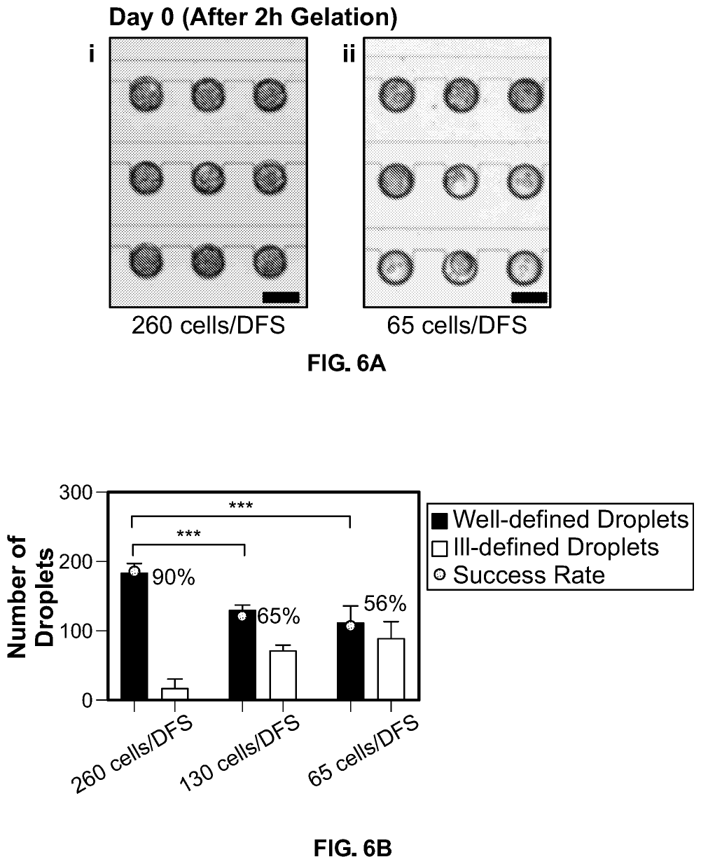

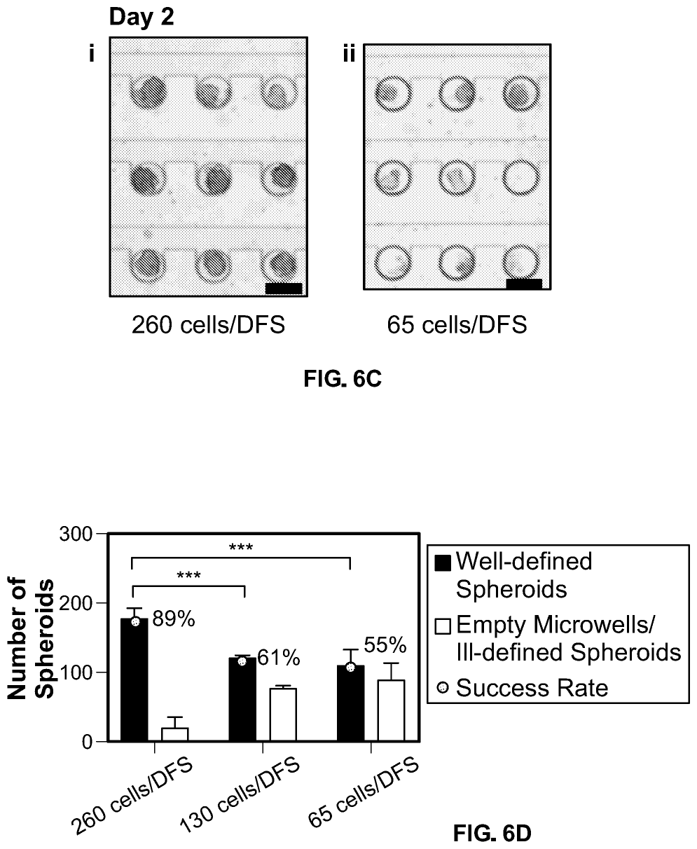

To characterize the formation and maintenance of DFSs in the microfluidic platform, we used suspensions with different cell densities of 1.2×10⁵, 2.5×10⁵, or 4.9×10⁵ cells/μL (corresponding to the final average number of hDFs in a DFS of 65, 130, and 260, respectively). At a high cell density (4.9×10⁵ cells/μL, 260 cells/DFS), every microwell in the microfluidics quadruplet was filled with a well-defined 100 μm-diameter cell-laden droplet that exhibited a uniform cell density (FIG. 6A (i)). At a lower cell density, however, a large number of wells were filled with ill-defined droplets with either smaller than microwell dimensions, or reduced cell density, as shown in brightfield images in FIG. 6A(ii). To characterize these trends quantitatively, we used two characteristics of the spheroid-on-a-chip platform, which we called, the Success Rate and Survival Rate. The Success Rate term characterized the fraction of wells with well-defined cell-laden droplets on day 0 and was defined as

$$Success Rate = \frac{Number of well - defined droplets on Day 0}{Total number of wells} \times 100 \%$$

The survival rate characterized the fraction of well-defined DFS remaining in the microfluidics quadruplet after 48-hour perfusion of the culture medium:

$$Survival Rate = \frac{Number of well - defined DFSs on Day 2}{Total number of wells} \times 100 \%$$

This trend is shown quantitatively in FIG. 6B. At a cell density in the suspension of 4.9×10⁵ cells/μL (or 260 hDFs/DFS), approximately 180 out of 200 microwells were filled with well-defined droplets, corresponding to a Success Rate of 90%. At a cell density of 1.2×10⁵ cells/μL (or 65 cells/DFS), however, the Success Rate was only 56%.

On Day 2, at cell density of 4.9×10⁵ cells/μL (or 260 hDFs/DFS), the DFSs formed from well-defined cell-laden droplets remained intact after purging the cell culture medium through the microfluidic device (FIG. 6C(i)). In contrast, at a cell density of 1.2×10⁵ cells/μL (or 65 hDFs/DFS), a notable number of empty microwells appeared on Day 2 (FIG. 6C(ii)). Notably, this effect was observed for the ill-defined cell-laden droplets formed on Day 0, whereas the rest of the microwells filled with well-defined droplets on Day 0 contained DFS on Day 2. As a result, the Survival Rate on Day 2 did not significantly differ from the Success Rate on Day 0 (FIGS. 6B and 6D) and was increasing with a higher cell density in the precursor solution suspension. Therefore, 1.2×10⁵ cells/μL (or 65 hDFs/DFS) demonstrated the highest Success Rate and Survival Rate and is selected as the optimal cell loading density.

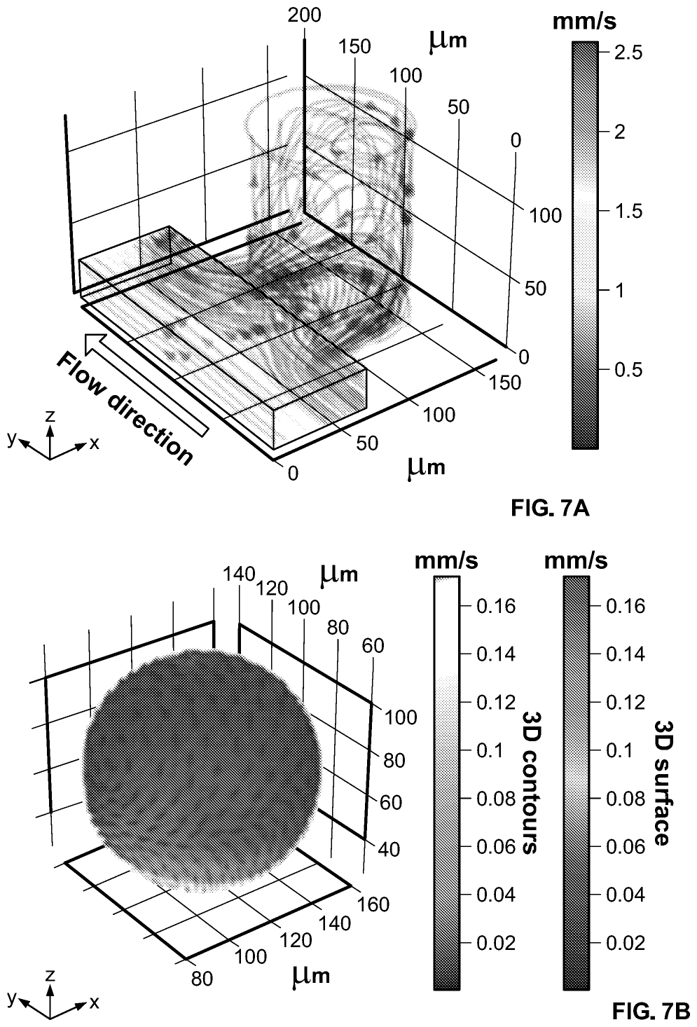

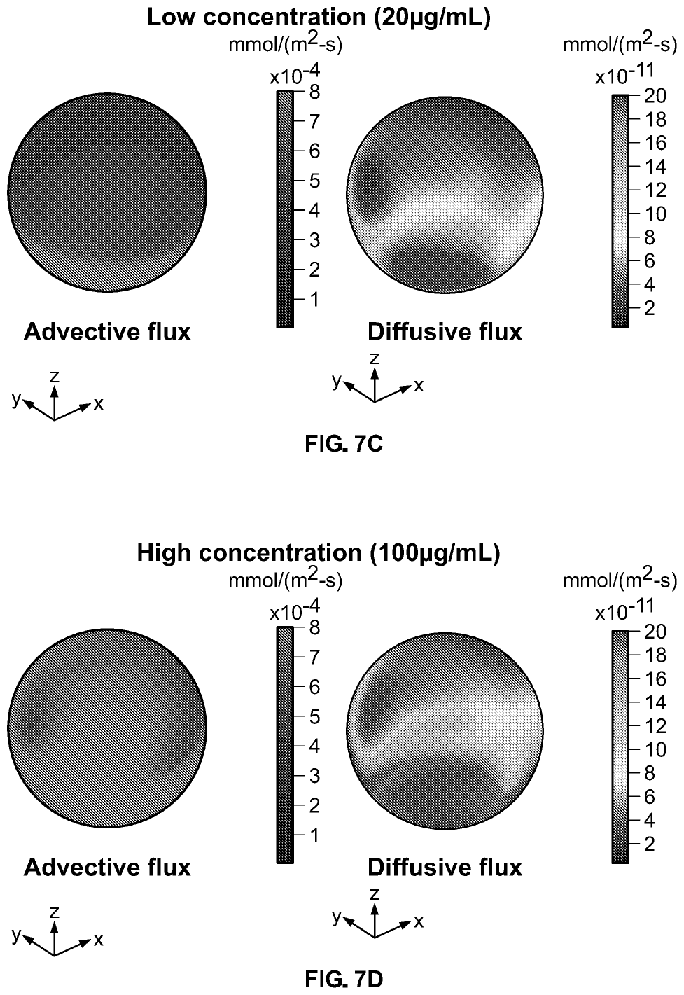

To study the fluid flow profile and the uptake of AIs by DFSs, a computational fluid dynamics (CFD) model was utilized. The CFD model was developed in COMSOL Multiphysics® by combining fluid flow and mass transport mode. We used the CFD model to simulate solute transport and uptake by the DFSs. The velocity field in a single microwell was computed, based on the pressure drop along a single well and visualized as flow streamlines (FIG. 7A). The CFD model predicted the velocity of the fluid surrounding DFSs in the microwells to be in the range of ˜2-16 μm/s (FIG. 7B), that is, the DFSs formed and were maintained under close-to-physiological interstitial and luminal flow. Next, we modelled advective and diffusive transport of low-molecular-weight solute molecules (such as vitamin C) to the DFSs localized in the microwells. The simulations were conducted for vitamin C (molecular weight 76.12 g/mol) flowed to the DFSs from solutions with concentrations of 20 and 100 μg/mL. There are two basic mechanisms in mass transport of biological molecules flowed with flowing liquids, namely, advective transport via interstitial flow due to the motion of the fluid, and diffusive transport that occurs due to the Brownian motion of molecules driven by the gradient of concentrations. Accounting for both mass transport mechanisms can improve our understanding of the delivery of Vitamin C to the spheroids. In the microfluidic platform, both transport mechanisms take place, as perfusion of molecules occurs due to the flow of culture medium containing AIs in the microchannel and microwells. The advection-diffusion fluxes were calculated and simulated under different solute concentrations (FIGS. 7C and D). Based on the solute penetration of the DFSs, we found that the change in its concentration from 20 to 100 μg/mL, increased its advective flux ˜4.8 times, while the diffusive flux was ˜2.4-fold higher. These results signified the importance of flow in the delivery of AIs to the DFSs.

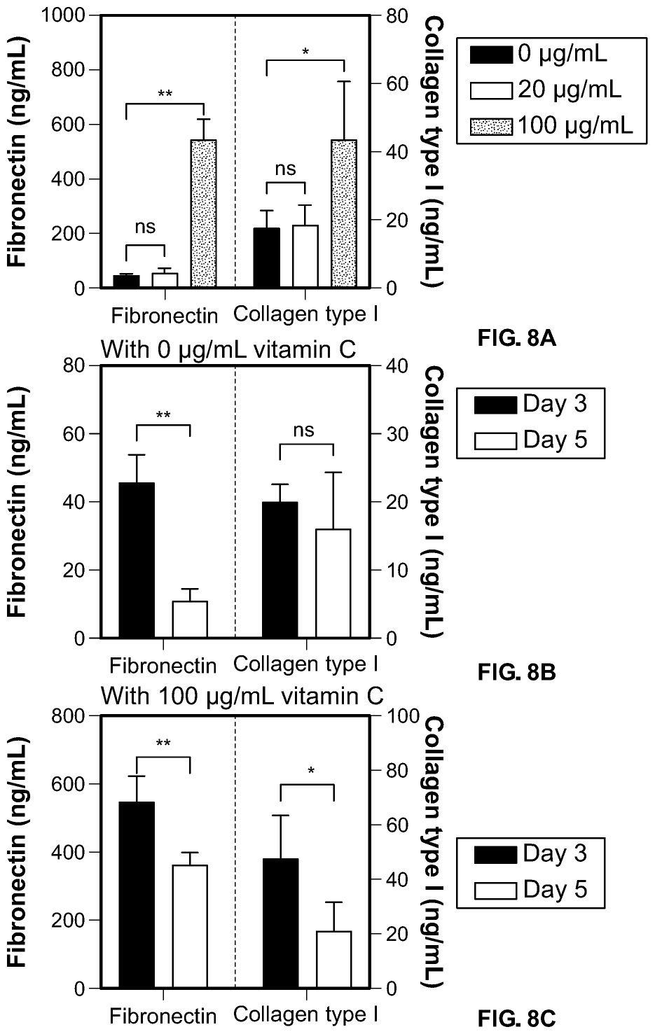

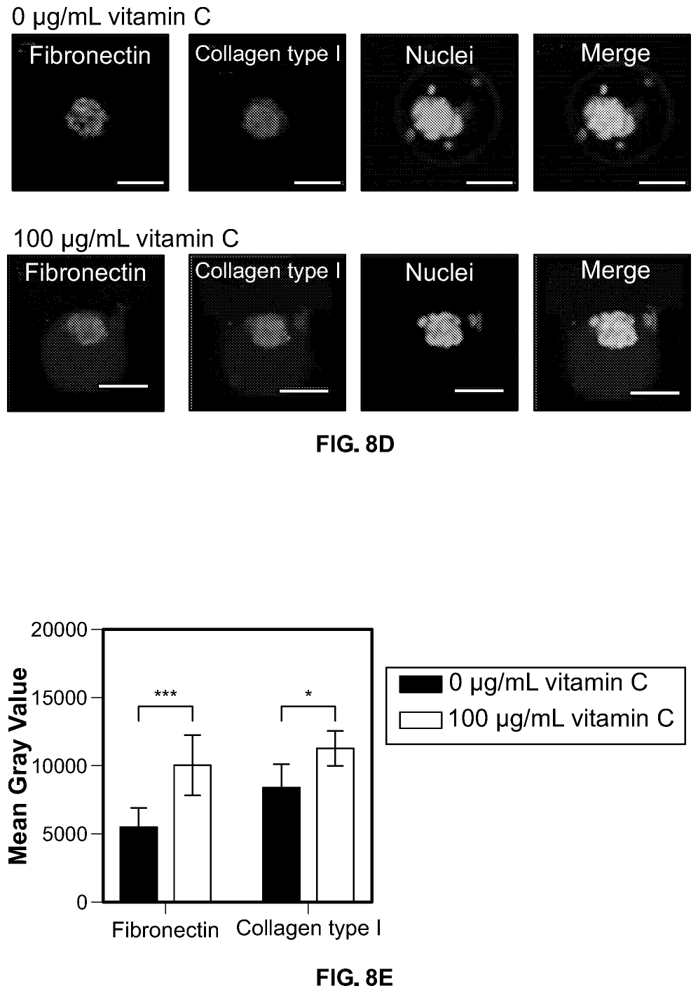

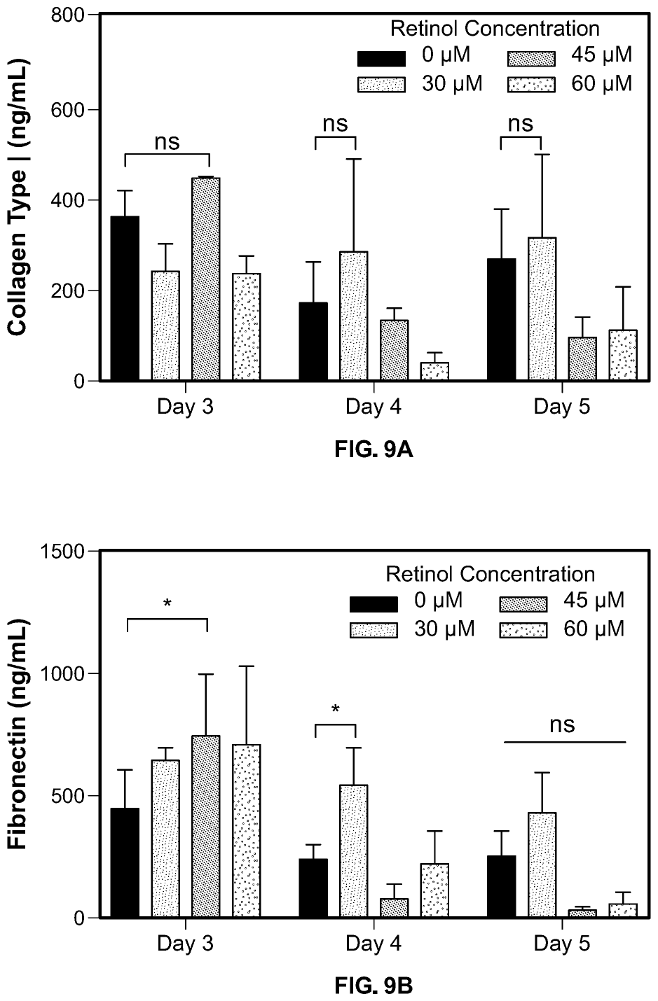

To demonstrate the capability of screening AIs, vitamin C and retinol were selected as the benchmark AIs for hydrophilic and hydrophobic AIs. FIG. 8A shows that an increased level of collagen type I and fibronectin secretion from DFSs was observed after treating 100 μg/mL vitamin C for 24-hours. FIGS. 8B and C show overtime change of collagen type I and fibronectin secreted by DFSs from 24 to 72 hours vitamin C treatment. FIGS. 8D and F show that the deposition of collagen type I and fibronectin in DFSs were increased as well after 72-hour treatment of 0 and 100 μg/mL vitamin C. FIG. 9 shows the preliminary study of retinol screening. Although a significant positive effect of retinol was not yet observed for collagen expression, a positive and significant effect for fibronectin production was observed a concentration of 30 and 45 μM, whereas a negative effect of retinol at a concentration of 60 μM was observed for the longer incubation time (day 5).

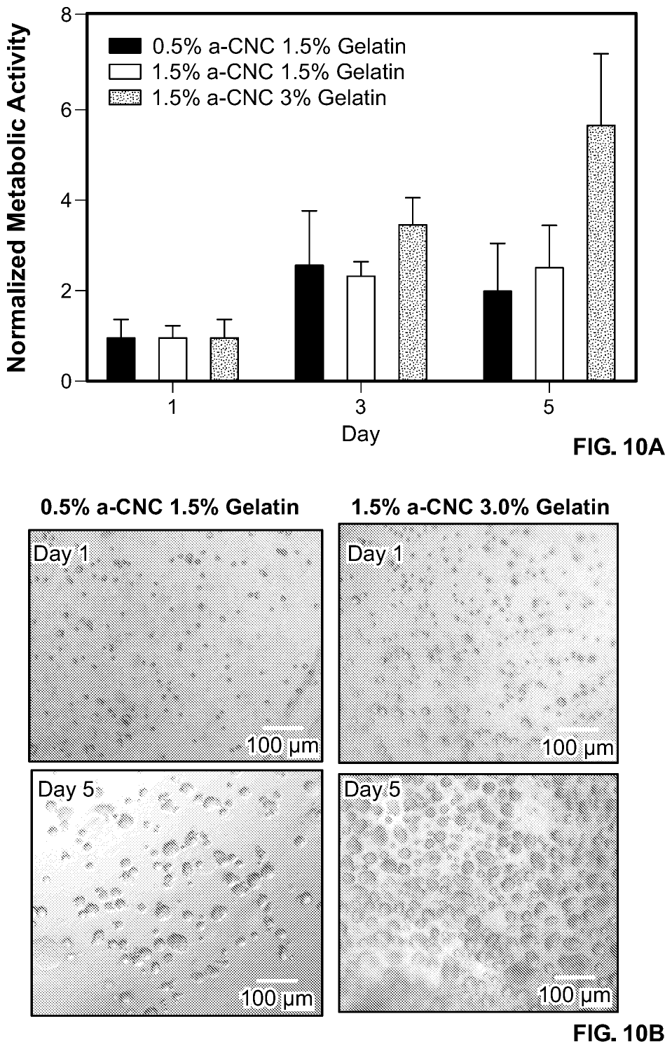

To optimize the EKGel II composition for the growth of keratinocytes, HaCaTs were encapsulated in different EKGel compositions (FIG. 10). In the EKGel with 1.5 wt % a-CNC and 3 wt % gelatin, HaCaTs exhibited the most growth in metabolic activity over time (FIG. 10A). Correspondingly, on Day 5, HaCaTs seeded on the EKGel with 1.5 wt % a-CNC and 3 wt % gelatin (FIG. 10C) are more confluent than HaCaTs seeded on the EKGel with 0.5 wt % a-CNC and 1.5 wt % gelatin (FIG. 10B)

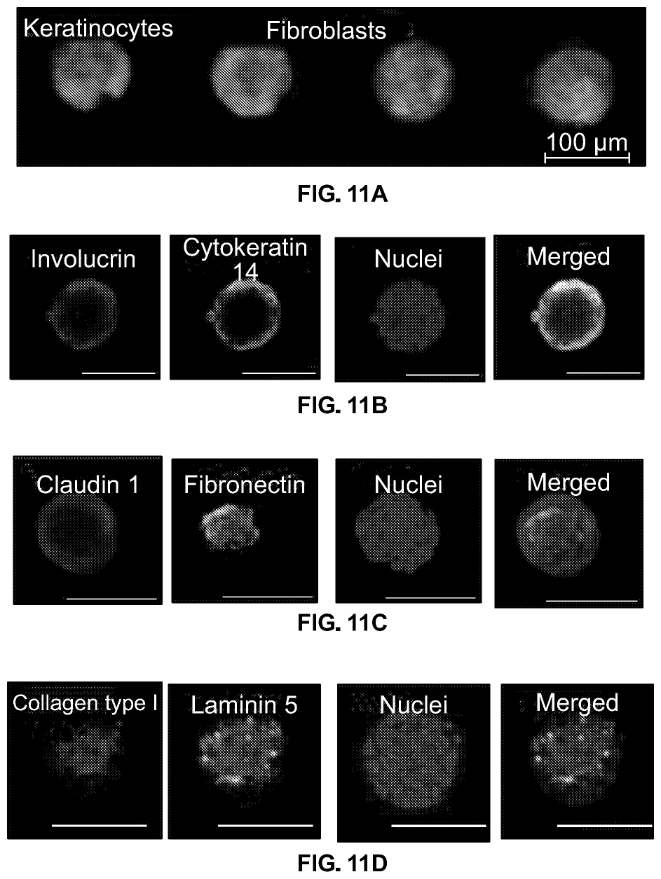

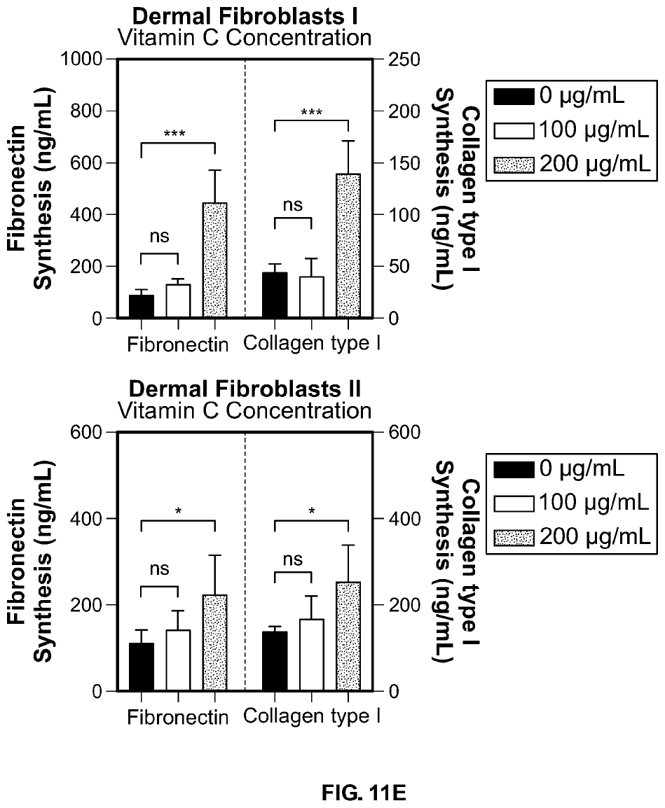

To characterize KDFSs, several fluorescent staining was conducted. FIG. 11A shows the structure of the KDFSs: the fibroblasts core is engulfed by a layer of keratinocytes shell. FIG. 11B shows the expression of the biomarkers for suprabasal keratinocytes and basal keratinocytes, involucrin and cytokeratin 14 respectively, on the epidermal layer of KDFSs. FIG. 11C shows the expression of tight cell junction, claudin 1, indicating the formation of a skin barrier. FIG. 11D shows the expression of laminin 5, indicating the formation of the epidermal-dermal junction. In FIG. 11B-C, collagen and fibronectin were stained as a reference to the location of dermal fibroblasts core. As a case study, we conducted a vitamin C screening on KDFSs formed from two different lineages of dermal fibroblasts (FIG. 11E). Due to the formation of the skin barrier in KDFSs, a higher concentration of vitamin C was required to stimulate the production of collagen and fibronectin in the 24-hour treatment compared to DFSs.

Taken together, the present approach is able to fabricate spheroids-based skin models, including a simple dermis model and a complex layered skin model. We verified the feasibility of both DFSs and KDFSs for the application of in vitro screening by screening different benchmark AIs. Our spheroids-based skin models are able to provide an effective response in a timely manner.

The skin spheroids-on-a-chip device disclosed herein is an effective and efficient solution to finding a replacement for live animal testing. Combining multicellular spheroids and microfluidic technology, fast growth of uniformly sized multicellular skin spheroids is achieved in a microfluidic array, allowing time-, cost-, and labor efficient screening of active ingredients. This platform can be further extended to effective screening of other substance such as pharmaceuticals or toxins.

The foregoing description of the preferred embodiments of the invention has been presented to illustrate the principles of the invention and not to limit the invention to the particular embodiment illustrated. It is intended that the scope of the invention be defined by all of the embodiments encompassed within the following claims and their equivalents.

What is claimed is:

1. A multicellular spheroid, comprising:
a core comprising first skin cells, wherein the core has an average diameter of about 10 μm to about 900 μm; and
a shell overlaying the core, the shell comprising second skin cells.

2. The multicellular spheroid of claim 1, wherein the first skin cells and the second skin cells independently comprise at least one of dermis cells, epidermis cells or hypodermis cells.

3. The multicellular spheroid of claim 2, wherein the dermis cells comprise one or more of dermal fibroblasts, myofibroblasts, keloid fibroblasts, dermal papilla cells, sebocytes or dermal dendritic cells.

4. The multicellular spheroid of claim 2 or 3, wherein the epidermis cells comprise one or more of keratinocytes, epidermal stem cells, myoepithelial cells, melanocytes, Langerhans cells or Merkel cells.

5. The multicellular spheroid of any one of claims 2 to 4, wherein the hypodermis cells comprise one or more of adipocytes, adipose derived stem cells, tenocytes, smooth muscle cells, gland stem cells or myoblasts.

6. The multicellular spheroid of any preceding claim, wherein the first skin cells comprise human dermal fibroblasts and the second skin cells comprise keratinocytes.

7. The multicellular spheroid of any preceding claim, wherein the shell has an average thickness of about 1 μm to about 30 μm.

8. The multicellular spheroid of any preceding claim, wherein the first skin cells form an aggregate.

9. The multicellular spheroid of any preceding claim, wherein the first skin cells are within a first hydrogel.

10. The multicellular spheroid of any preceding claim, wherein the second skin cells are within a second hydrogel.

11. The multicellular spheroid of any preceding claim, wherein the shell is adhered to the core.

12. The multicellular spheroid of any preceding claim, wherein the first skin cells are contracted.

13. A multicellular spheroid system, comprising:
a plurality of multicellular spheroids, each multicellular spheroid comprising:
a core comprising first skin cells, wherein the core has an average diameter of about 10 μm to about 900 μm; and
a shell overlaying the core, the shell comprising second skin cells,
wherein the plurality of multicellular spheroids are arranged in an array of microwells.

14. The system of claim 13, wherein the array of microwells is within a microfluidic device.

15. The system of claim 13 or 14, wherein each microwell has a cylindrical shape.

16. The system of any one of claims 13 to 15, wherein each microwell has a diameter of about 50 μm to about 1000 μm.

17. The system of any one of claims 13 to 16, wherein the array of microwells is connected with a supplying channel.

18. The system of any one of claims 13 to 17, wherein each of the plurality of multicellular spheroids has an average diameter that is within about ±1 μm to about ±10 μm of each other.

19. The system of any one of claims 13 to 18, wherein each of the plurality of multicellular spheroids has a spherical shape, a cylindrical shape or a disc shape.

20. A microfluidic-based process for preparing multicellular spheroids, comprising:
flowing a first skin cell-laden suspension into microwells and forming cell-laden droplets and inducing gelation to form gelled cell-laden droplets;
supplying a first aqueous culture medium to the microwells to induce aggregation of the gelled cell-laden droplets and form compacted spheroids;
flowing a second skin cell-laden suspension through the microwells to surround the compacted spheroids and induce gelation; and
supplying a second aqueous culture medium to the microwells, wherein the second aqueous culture medium induces formation of the multicellular spheroids, each of the multicellular spheroids comprising a core comprising first skin cells and a shell comprising second skin cells.

21. The process of claim 20, wherein the first skin cell-laden suspension comprises first skin cells suspended in an aqueous fluid, or
wherein the first skin cell-laden suspension comprises first skin cells suspended in a hydrogel precursor solution.

22. The process of claim 20 or 21, wherein preparing the first skin cell-laden suspension comprises:
combining first skin cells with a first physiologically compatible hydrogel precursor solution.

23. The process of claim 22, wherein the first physiologically compatible hydrogel comprises constituents that provide a biomimetic extracellular environment to assist in formation of the compact spheroids from the first cells and to support metabolic activity of the compact spheroids.

24. The process of claim 22 or 23, wherein the first physiologically compatible hydrogel precursor solution comprises about 0.3 wt % to about 2 wt %, or about 0.5 wt % aldehyde-functionalized cellulose nanocrystals (a-CNCs) and about 0.6 wt % to about 4 wt %, or about 1.5 wt % gelatin.

25. The process of any one of claims 20 to 24, further comprising:
flowing the first cell-laden suspension through microchannels into the microwells.

26. The process of any one of claims 20 to 25, wherein the second skin cell-laden suspension comprises second skin cells suspended in an aqueous fluid, or
wherein the second skin cell-laden suspension comprises second skin cells suspended in a hydrogel precursor solution.

27. The process of any one of claims 20 to 26, wherein preparing the second skin cell-laden suspension comprises:
combining second skin cells with a second physiologically compatible hydrogel precursor solution.

28. The process of claim 27, wherein the second physiologically compatible hydrogel comprises constituents that provide a biomimetic extracellular environment to assist in formation of the shell containing the second skin cells surrounding the compact spheroids and to support metabolic activity of the shell and maintain structural integrity of the hybrid multicellular spheroids.

29. The process of claim 27 or 28, wherein the second physiologically compatible hydrogel precursor solution comprises about 0.3 wt % to about 2 wt %, or about 1.5 wt % aldehyde-functionalized cellulose nanocrystals (a-CNCs) and about 0.6 wt % to about 4 wt %, or about 3.0 wt % gelatin.

30. The process of any one of claims 20 to 29, further comprising:
flowing the second skin cell-laden suspension through microchannels into the microwells.

31. The process of any one of claims 20 to 31, wherein forming the cell-laden droplets comprises a self-digitization process to fill each microwell with the first skin cell-laden suspension.

32. The process of any one of claims 20 to 32, wherein the first aqueous culture medium is conFIG.d to induce contraction of the cell-laden droplets to form the compacted spheroids.

33. The process of any one of claims 20 to 33, wherein a surface of each of the compacted spheroids comprises free space between the surface and walls of the microwells.

34. The process of any one of claims 20 to 33, further comprising:
pre-wetting the microwells with a fluid immiscible to the first skin cell-laden suspension.

35. The process of any one of claims 20 to 34, further comprising:
after forming the gelled cell-laden droplets, flowing a fluid immiscible to the first skin cell-laden suspension through microchannels connected with the microwells for displacing any extra first skin cell-laden suspension located therein,
after forming the compacted spheroids, flowing a fluid immiscible to the first aqueous culture medium through microchannels connected with the microwells for displacing any extra first aqueous culture medium located therein, and
after flowing the second skin cell-laden suspension through the microwells, flowing a fluid immiscible to the second skin cell-laden suspension through microchannels connected with the microwells for displacing any extra second skin cell-laden suspension located therein.

36. The process of claim 34 or 35, wherein the immiscible fluid comprises at least one of a biocompatible oil, an organic liquid, an organic oil, or a fluorinated oil.

37. The process of claim 36, wherein the fluorinated oil comprises about 0.1 wt % to about 1.0 wt %, or about 0.5 wt % of a fluorosurfactant.

38. The process of any one of claims 20 to 37, wherein one or more process steps is conducted at a temperature of about 37° C. under an atmosphere of about 5% CO₂.

39. The process of any one of claims 20 to 34, wherein each of the microwells has a diameter of about 50 μm to about 1000 μm.

40. The process of any one of claims 20 to 39, wherein the first skin cells and the second skin cells independently comprises at least one of dermis cells, epidermis cells or hypodermis cells.

41. The process of claim 40, wherein the dermis cells comprise one or more of dermal fibroblasts, myofibroblasts, keloid fibroblasts, dermal papilla cells, sebocytes or dermal dendritic cells.

42. The process of claim 40, wherein the epidermis cells comprises one or more of keratinocytes, epidermal stem cells, myoepithelial cells, melanocytes, Langerhans cells or Merkel cells.

43. The process of claim 40, wherein the hypodermis cells comprise one or more of adipocytes, adipose derived stem cells, tenocytes, smooth muscle cells, gland stem cells or myoblasts.

44. The process of any one of claims 20 to 43, wherein the first skin cells are human dermal fibroblasts, and the second skin cells are human keratinocytes cells.

45. The process of any one of claims 20 to 44, wherein the first skin cell-laden suspension comprises about 5×10³ cells/μL to about 5×10⁶ cells/μL, or about 4.9×10⁵ cells/μL dermal fibroblast cells.

46. The process of any one of claims 20 to 45, wherein the second skin cell-laden suspension has a cell/gel mass ratio of about 100:0 to about 50:50, or about 75:25.

47. The process of any one of claims 20 to 46, wherein the first skin cell-laden suspension flows from a preparation reservoir through microchannels to the microwells at a flow rate of about 0.01 mL/hr to about 0.30 mL/hr.

48. The process of any one of claims 20 to 47, wherein the second skin cell-laden suspension flows from a preparation reservoir through microchannels to the microwells at a flow rate of about 0.1 mL/hr to about 1 mL/hr.

49. The process of any one of claims 20 to 48, wherein at least one of the first aqueous culture medium or the second aqueous culture medium flows from a preparation reservoir through microchannels to the microwells at a flow rate of about 0.001 mL/hr to about 0.5 mL/hr.

50. Multicellular spheroids comprising a core comprising first skin cells and a shell comprising second skin cells produced by the process of any one of claims 20 to 49.
